(19) United States
(12) Patent Application Publication
(10) Pub. No.: US 2020/0129438 A1
(43) Pub. Date: Apr. 30, 2020

(54) FINISHED PHARMACEUTICAL DOSAGE FORM COMPRISING A LOW DOSE/HIGH POTENCY ACTIVE PHARMACEUTICAL INGREDIENT AND ONE OR MORE EXCIPIENTS

(71) Applicant: David F. Erkoboni, Pennington, NJ (US)

(72) Inventor: David F. Erkoboni, Pennington, NJ (US)

(73) Assignee: Edenbridge Pharmaceuticals, LLC, Parsippany, NJ (US)

(21) Appl. No.: 16/176,888

(22) Filed: Oct. 31, 2018

(51) Int. Cl. A61K 9/20; A61K 31/7048; A61K 45/06

(52) U.S. Cl. CPC A61K 9/2095; A61K 9/2054; A61K 45/06; A61K 9/2013; A61K 31/7048; A61K 9/2059

(57) ABSTRACT

The present application discloses a finished pharmaceutical dosage form comprising a low dose/high potency active pharmaceutical ingredient and one or more excipients. The active pharmaceutical ingredient in the present application has been blended and subsequently milled with at least one excipient before preparing the finished pharmaceutical dosage form. The disclosed finished dosage form promotes content uniformity and acceptable and reproducible dissolution rate and extent. Finished dosage forms of the present invention may comprise any active pharmaceutical ingredient; however, low solubility and/or high potency active pharmaceutical ingredients will particularly benefit. Among the low solubility and/or high potency active pharmaceutical ingredients, those with needle-like structures are even more particularly well suited for incorporation into the disclosed finished dosage forms. A finished dosage form containing ivermectin is one non-limiting example of a finished dosage form contemplated by the disclosure herein. Hormonal steroids are also particularly well suited for the invention described herein.

### BACKGROUND OF THE INVENTION

A recent trend in the pharmaceutical industry is the use of active pharmaceutical ingredients (“APIs” or in the singular form “API”) with low solubility in water (“low solubility” is defined as 1 part in 1,000 parts or less than 1 part in 1,000 parts) in finished dosage drug products. Another trend can be seen in the use of APIs with lower therapeutic doses, meaning the selection of APIs with high potency (“high potency” is defined as requiring 25 mg or less of an API in a finished dosage form to be therapeutically effective). For purposes of this application, “high potency” and “low dose” may be used interchangeably. The combination of high potency and low solubility in a single API results in significant challenges for pharmaceutical formulation scientists because these characteristics tend to cause problems in achieving acceptable content uniformity and also cause problems in attaining required dissolution performance, in particular, acceptable and reproducible dissolution rate. As used throughout this application, “acceptable content uniformity” means a product that meets the current United States Pharmacopeia (USP) acceptance value, and “acceptable dissolution performance” means routinely passing dissolution criteria or Q value(s) as set forth in the USP and/or the United States Food and Drug Administration Dissolution Database. The effect of both formulation ingredients (excipients and API) and manufacturing process(es) on content uniformity and dissolution performance must be considered when formulating and processing a low dose API with low solubility.

Using finely divided powdered materials, both APIs and excipients, is generally considered beneficial for achieving superior dissolution, but the use of such materials can create challenges for achieving acceptable content uniformity with simple, inexpensive processing techniques such as dry blending. Even when acceptable content uniformity is achieved, however, the finely divided powder blends can have a negative impact on several downstream processes. For example, fine powders flow poorly and, in general, have significantly lower bulk density than coarser powders resulting in poor control of the downstream tableting, encapsulation, or powder filling processes. Because of the aforementioned flow and density concerns, agglomeration methods are commonly needed as additional processing steps, which present additional challenges for a pharmaceutical formulation scientist. Other downstream processes, such as wet granulation or dry granulation, can be negatively impacted by powder blends consisting of fine particle size materials.

The most common methods of preparing API-excipient mixtures, for use in the preparation of solid oral finished dosage forms, are wet granulation (high shear and fluid bed processing), dry granulation (slugging and roller compaction), and dry blending. As will be explained in greater detail below, both wet and dry granulation processes are significantly more expensive than dry blending. Other, more specialized processes such as spray drying, hot melt extrusion, hot melt granulation, and extrusion/spheronization can be used, but these processes are even more costly than the wet or dry granulation processes.

Granulation processes are commonly used within the pharmaceutical industry to improve flow, increase density, improve content uniformity, and possibly increase the rate and extent of dissolution. During these processes, however, primary particles of one or more of the dosage form components are combined and bound together either (i) in a wet process with fluid and a wet binder or (ii) in a dry process with compaction force and a dry binder. This combining of primary particles during granulation limits the movement of the API within the granulation. This immobility can help promote—but does not guarantee—content uniformity within the finished dosage form. If performed correctly, the granulation processes can promote content uniformity and, in some cases, rapid and more complete dissolution; however, as mentioned above, these processes are significantly more expensive than dry blending and do not guarantee content uniformity. For example, even if performed correctly, poor content uniformity can occur during (i) wet granulation due to migration of the drug substance during the drying step and (ii) dry granulation due to the production of a large amount of fines. Regardless of the granulation process used, the API needs to be uniformly distributed throughout the excipient particles; otherwise, it can result in poor blend uniformity and, subsequently, poor content uniformity.

Granulation processes (both wet and dry) have other significant downsides. They are both labor and manufacturing time intensive, and they can be very energy intensive. They also have a high capital equipment cost. In rank order, the fluid bed granulation process is more expensive than the high shear granulation process, which is more expensive than the roller compaction process, which is more expensive than the slugging process.

In dry blending, materials are typically screened and blended in a low shear blender, such as a V-shaped or conical blender. The dry blending process, as conventionally applied, will not consistently manufacture a uniform powder mixture of a low dose API, regardless of the API solubility. There is nothing inherent in the process of blending materials that can guarantee a uniform distribution of the API resulting in consistent content uniformity of a blend containing a low dose API. An API with low solubility adds to the overall formulation challenge because (1) the surface area of an API that is not finely divided is lower and provides less exposure to the dissolution media and (2) the API is not uniformly mixed with the excipients and may not benefit from the intended solubilizing benefits such as wetting, fluid absorption, and rapid dissolution.

Dry blending is by far the least expensive process for producing API-excipient mixtures. It is the least labor- and energy-intensive and also requires the least amount of manufacturing time. Dry blending also requires an inexpensive capital equipment train when compared to the equipment train required for granulation processes. Given these advantages, it is highly desirable to make products using a dry blending manufacturing process.

Most, if not all pharmaceutical materials, APIs and excipients alike, are produced using a drying step as the last or near-to-the-last manufacturing step. Drying processes are used for both APIs and excipients to remove water or other organic solvents; however, drying processes can produce unintended, weakly bound secondary particles comprising multiple primary particles. This is problematic because weakly bound secondary particles are larger and have less surface area, which can reduce interaction with the excipients and can result in a lower dissolution rate. Milling imparts mechanical energy and is typically used to break secondary particles that are weakly held together or bound by mechanisms such as electrostatic forces, crystal bridges, and/or mechanical interlocking.

Milling ingredients individually, API or excipient, prior to blending, can reduce the size of large primary particles or can disrupt the bonds creating unintended secondary particles. In the case of the bonds resulting from electrostatic forces or mechanical interlocking, the secondary particles can reform—and likely will reform—after the milling step. In the case of secondary particles formed by unintended crystal bridges, the bonds will likely not reform after milling. Further, the smaller primary particles created by milling will have a higher surface energy that can be reduced by forming secondary particles in a similar manner to the phenomenon described for the reformation of secondary particles. It is postulated that milling ingredients individually prior to formulation and blending may not result in the desired characteristics intended by the pharmaceutical formulator.

Milling ingredients individually can also result in significant loss of certain ingredients. For example, API loss can result from airborne dust lost to the dust collector or by adhering to the process equipment surfaces. Ivermectin is an example of an API that has significant yield loss resulting from airborne dust lost to the dust collector and material adhering to the process equipment surfaces.

U.S. Pat. No. 7,976,872 B2 discloses a process for distributing an API in an excipient by: (i) providing a first layer of excipient; (2) providing a layer of API on the first layer of excipient; (3) passing the two layers through a static mixer; and (4) discharging the blended mixture from the static mixer. This patent also notes that the disclosed process is useful for blending low dose APIs that have a propensity to stick to processing equipment with excipients. The process disclosed in the present invention does not involve layering of API and excipients or a static mixer; rather, the present invention comprises a formulation comprised of API and at least one excipient, a dry blending step, a milling step and an optional second blending step. The present invention may be practiced with diffusion mixers or convection mixers and pharmaceutically acceptable mills such as impact mills, cutting mills, and screening mills. The formulation and process of the present invention will be described in more detail below.

U.S. Pat. No. 7,976,872 also describes prior art “[c]onventional methods of dispersing a high potency, low dose drug into a physically inert material that is subsequently processed into a solid oral dosage form [that] have involved preparing a premix or granulation in which a portion of one or more of the physiologically inert materials is first combined with the physiologically active compound using a low shear mixing device, such as a twinshell blender, a double cone blender, or a drum roll mixer.” Col. 1 ll. 16-23. U.S. Pat. No. 7,976,873 further describes that in these conventional methods, the premix or granulation is passed through a mill to enhance homogeneity of the mixture, and the milled material can optionally be blended with other materials to form a final product mixture that is compressed into a tablet, filled into a capsule, or otherwise incorporated into a solid oral dosage form. Col. 1 ll.23-29. The present invention differs from these conventional methods described in U.S. Pat. No. 7,976,873 because the premixes described in those conventional methods use small portions of the final blend; whereas, the present invention does not use a small quantity of the excipient base to prepare a pre-mix; rather, in this invention, a significant portion of the excipient base is used, with 50% or greater used in a preferred embodiment and up to 100% of the excipient base used in an even more preferred embodiment.

### BRIEF SUMMARY OF THE INVENTION

The present invention relates to a formulation of finished pharmaceutical dosage forms including powders, tablets, and capsules, comprised of at least one API and at least one excipient wherein the API and the excipient are present in a substantially uniform mixture within the finished dosage form. In a preferred embodiment of the invention, the API is a high potency API. In another preferred embodiment, the API is a high potency API with low solubility. Ivermectin and hormonal steroids are exemplary APIs contemplated by the present invention. The finely divided mixture of API and excipients is achieved by milling a blend of the API and the material(s) comprising the pharmaceutical excipient base. More specifically, milling APIs that have needle-shaped primary particles, such as ivermectin, is particularly beneficial because the milling fractures the needles such that their long dimension now more closely approximates their shorter dimension, which results in a more cubic particle which promotes better content uniformity. The finished dosage form prepared from the milled blend has a substantially uniform distribution of API and a rapid dissolution rate when compared to dissolution without milling.

An object of the invention is to provide a dry blending process for manufacturing a finished dosage form comprising a low dose API having acceptable content uniformity, as well as rapid and complete dissolution. Any low dose/high potency API will benefit from this invention. Low solubility and low dose/high potency APIs will particularly benefit from this invention. A particularly preferred API of the present invention is ivermectin. Hormonal steroids are also well suited for the present invention.

The present invention uses a dry blending and milling process to result in a fine division and intimate dispersion of all ingredients within the final composition. The finely divided dispersion results in acceptable content uniformity as well as acceptable dissolution performance of the prepared finished dosage form. The dry blending process uses the least amount of manufacturing time and energy of all pharmaceutical processes and the required equipment is the least expensive.

The present application discloses a process to create a substantially uniform distribution of a low dose API in a pharmaceutical excipient base (containing one or more excipients) to promote content uniformity as well as rapid dissolution of the finished dosage form. The pharmaceutical excipient base comprises any inert material, functional or non-functional. Such inert materials include but are not limited to binders/fillers, disintegrants, lubricants, stabilizers/preservatives, and colorants. The low dose API is blended with the excipient base followed by a milling step using a pharmaceutically acceptable mill. Examples of acceptable mixers include diffusion mixers or convection mixers. Examples of pharmaceutically acceptable mills include impact mills, cutting mills, and screening mills. After the milling step, the API-excipient mixture may be subjected to an option re-blended and lubricated, filled into packets, compressed into tablets, or filled into appropriately sized hard gelatin capsules. This process allows for a substantially uniform distribution of not only the API but also any functional pharmaceutical excipients. The powders, tablets, or capsules produced using this method have a substantially uniform distribution of API, as well as rapid dissolution, when they may not otherwise.

### DETAILED DESCRIPTION OF THE INVENTION

The present invention relates to a formulation of a finished dosage form, which may be a powder, tablet, or capsule comprised of at least one API and at least one excipient wherein the API and the excipient are present in a finely divided mixture within the finished dosage form. In a preferred embodiment of the invention, the API is a high potency API. In another preferred embodiment, the API is a high potency API with low solubility. Ivermectin is one non-limiting example of an API particularly well suited for this invention. Hormonal steroids are also well suited for this invention. A combination of hormonal steroids in one finished dosage form is expressly contemplated by the disclosure herein.

The disclosed process comprises a dry blending step, followed by a milling step, to prepare a substantially finely dispersed, uniform powder blend, which can then be further processed into the finished dosage form including tablets, capsules, and powders. The powder blend is comprised of the API and at least one pharmaceutically acceptable inert ingredient, commonly referred to as an excipient.

Finished dosage forms prepared from low dose APIs generally require time, labor, and energy-intensive processes to achieve a product with acceptable content uniformity. The difficulties are only exaggerated when the low dose API also has low solubility because the process used to enhance flow, such as wet and dry granulation, may not have a positive effect on dissolution. The dosage forms prepared using the formulation and process of this invention require only a dry blending and milling process step. This is a streamlined process train, with respect to time, labor, and energy, as compared to granulation processes (e.g., wet granulation and dry granulation) and other, more specialized processes including but not limited to spray drying, hot melt extrusion, hot melt granulation, and extrusion/spheronization.

The dry blending and milling steps of this invention are used to prepare a powder blend having a substantially finely dispersed, uniform distribution of the API within the excipient base wherein the final blend is comprised of primary and tightly bound secondary particles as they were intended by the material manufacturers. The creation of a final blend comprised of primary and secondary particles is a significant advantage when compared to the granulation processes.

Milling of the API, together with the excipient base, has particular advantages for APIs having a needle-like structure. Acceptable blend uniformity can be achieved with APIs having a needle-like structure, but such APIs often do not flow well on processing equipment (e.g., a compression machine, encapsulator, or sachet/packet filler), which may result in poor content uniformity. The present invention solves this problem as a result of the change in physical form of the API as it is milled. Co-milling the low dose poorly soluble API having a needle-like structure and the excipient base not only breaks the secondary particles into primary particles, but it also fractures the needles such that their long dimension now more closely approximates their shorter dimension. When these dimensions more closely approximate each other, better flow of the API is achieved during encapsulation, sachet filling, or tableting, which results in a more substantially uniform finished dosage form.

Any pharmaceutically acceptable excipient can be used to practice this invention. Examples of such pharmaceutical excipients that can be used to practice this invention include, but are not limited to: (i) binders/fillers such as starch, microcrystalline cellulose, lactose, and dicalcium phosphate; (ii) disintegrants such as starch, croscarmellose sodium, sodium starch glycolate, and cross-linked polyvinyl pyrrolidone; (iii) glidants such as silica and talc; (iv) lubricants such as stearic acid, magnesium stearate, and sodium stearyl fumarate; (v) colorants such as FD&C lake pigments; (vi) flavoring agents such as natural and artificial flavors; (vii) preservatives such as benzoic acid; (viii) antioxidants such as butylated hydroxyanisole, and butylated hydroxytolulene; and (ix) pH modifiers such as citric acid and fumaric acid.

The disclosed process comprises a first dry blending step, followed by a milling step, which is in turn is optionally followed by a second blending step. The first blending step is used to ensure that the API and excipients in the powder mix are sufficiently distributed. The milling step is used to shear all ingredients into the primary particles or tightly bound secondary particles as they were intended to be by the material manufacturers and intimately disperse them in a substantially uniform distribution. The last step, an optional step, is a second blending step. In this second blending step, a lubricant may be added and blended to the milled API-excipient mixture. The disclosed process—with or without the optional second blending step—results in a fine dispersion of the ingredients exhibiting good blend uniformity resulting in a finished dosage form exhibiting acceptable content uniformity and facilitates a more rapid dissolution of the prepared finished dosage form.

In a preferred form of the invention, the API is blended and milled with at least 90-100% of the total excipient quantity of the dosage form formulation. Using 90-100% of the excipient quantity of the dosage form formulation ensures the greatest amount of excipient is present in the finished dosage form in the particle configuration intended by the material manufacturer. Particles intended by the manufacturer to be primary will be as intended. Particles intended to be tightly bound secondary particles will be as intended. Primary and secondary particles will not be bound together unintentionally by weak electrostatic forces, weak crystal bridges, or weak mechanical bonding as would be seen with other processing methods such as granulation. These weakly bound particles of API or excipient seen in other methods can create challenges in content uniformity and dissolution in the finished dosage form, a disadvantage not seen in the present invention.

The invention is intended to help with content uniformity and dissolution of finished dosage forms with low dose APIs. The dose of the API does not define the percent of the API in the finished dosage form, though the dosage form needs to have a reasonable mass to be viable for administration. Relatedly, on the opposite end of the spectrum, the percentage of API must not be so small such that the total mass and size of the finished dosage form is too large for administration by a patient in need thereof. A dosage form containing as much as 50% API is contemplated by the invention. Although the present invention achieves acceptable content uniformity and acceptable dissolution performance at a 1:1 ratio of API to excipient(s)s (“API:excipient(s)”), higher ratios are more typical and preferred. In a preferred form of the invention, the API is blended and milled with an amount of excipient base from at least equal to the weight of the API to five (5) times the weight of the API (i.e., a 1:1-1:5 API:excipient ratio). In a more preferred form of the invention, the API is blended and milled with an amount of excipient base from greater than five (5) times weight of the API to twenty (20) times the weight of the API (i.e., a >1:5-1:20 API:excipient ratio). In the most preferred form of the invention, the API is blended and milled with an amount of excipient base from greater than twenty (20) times the weight of the API (i.e., a >1:20 API:excipient ratio) but not so small of a ratio such that the dosage form is too large to be administered.

Although any pharmaceutically acceptable excipient can be used in the present invention by itself or in combination with other pharmaceutically acceptable excipients, a preferred form of the invention contains at least one pharmaceutically acceptable excipient that promotes rapid water uptake, including but not limited to microcrystalline cellulose or starch. An even more preferred form of the invention contains at least one pharmaceutically acceptable excipient that promotes rapid water uptake, including but not limited to microcrystalline cellulose or starch, and does not contain an excipient that deforms by brittle fracture. A non-limiting example of an excipient that deforms by brittle fracture is dicalcium phosphate. A yet even more preferred embodiment contains at least one pharmaceutically acceptable excipient that deforms plasticly and promotes rapid water uptake, including but not limited to microcrystalline cellulose or partially pregelatinized starch, and does not contain an excipient that deforms by brittle fracture.

Any pharmaceutically acceptable mill can be used to practice this invention. Pharmaceutically acceptable mills include impact mills, cutting mills, and screening mills. One factor influencing the performance of the blending/milling process of this invention is the size of the screen used in the milling process. Exemplary screen sizes may range from 500 μm-2000 μm.

### Example 1

The formula and process of one exemplary embodiment of the present invention is described below.

| | Mg/tablet | Percent (%) |
|---|---|---|
| Active Pharmaceutical Ingredient | | |
| Ivermectin | 2.00 | 2.00 |
| Excipient Base | | |
| Microcrystalline cellulose 100 μm | 52.06 | 52.06 |
| Microcrystalline cellulose 20 μm | 26.03 | 26.03 |
| Pregelatinized Starch | 19.54 | 19.54 |
| Citric acid | 0.02 | 0.02 |
| Butylated hydroxyanisole | 0.02 | 0.02 |
| FD&C blue lake | 0.08 | 0.08 |
| Mg stearate | 0.25 | 0.25 |
| Total | 100.00 | 100.00 |

All materials (ivermectin, microcrystalline cellulose 100 μm, microcrystalline cellulose 20 μm, pregelatinized starch, citric acid, butylated hydroxanisole, FD&C Blue, and magnesium stearate are passed through a 40 mesh screen. Using geometric dilution principles, one third of the total microcrystalline cellulose is charged into a v-blender followed by the ivermectin and pregelatinized starch. The materials are mixed for 5 minutes. Citric acid, butylated hydroxyanisole and FD&C blue lake are added to the mixer followed by another third of the total microcrystalline cellulose. The mixture is blended for 5 minutes. The remaining amount of microcrystalline cellulose is added to the blender and blended for 10 minutes. The materials are discharged from the blender and the blend is milled using a comminution mill (Fitzpatrick J-type) with hammers forward, at medium speed, using a 25 mesh screen (1522-0027). The milled blend is charged back into the v-blender followed by magnesium stearate. The mixture is blended for 5 minutes. The final blend is discharged from the blender and charged into the hopper of a tablet press. Tablets are compressed using 0.2717 in. diameter, flat face, beveled edge tooling to a hardness of 4 kp.

Content uniformity results are shown in the table below. All values are well within acceptable limits.

| | | Ivermectin | Ivermectin |
|---|---|---|---|
| Sample | Tablet Weight | Recovered | Recovered |
| Tablet No. | (mg) | (mg) | (%) |
| 1 | 99.12 | 1.98 | 99.1 |
| 2 | 98.94 | 2.01 | 100.7 |
| 3 | 98.80 | 1.99 | 99.6 |
| 4 | 98.88 | 2.01 | 100.5 |
| 5 | 98.87 | 1.94 | 97.2 |
| 6 | 100.14 | 2.03 | 101.4 |
| 7 | 99.40 | 2.06 | 102.8 |
| 8 | 100.67 | 1.93 | 96.7 |
| 9 | 99.97 | 1.98 | 98.8 |
| 10 | 100.57 | 1.95 | 97.6 |
| Average | 99.50 | 1.99 | 99.3 |
| Maximum | 100.67 | 2.06 | 102.8 |
| Minimum | 98.80 | 1.93 | 96.7 |
| RSD % | 0.7 | 2.0 | 2.0 |

Dissolution results are shown in the table below.

| Time (min) | Percent Dissolved (%) |
|---|---|
| 0 | 0 |
| 5 | 67 |
| 10 | 86 |
| 20 | 97 |
| 30 | 101 |
| 45 | 103 |
| 60 | 104 |

What is claimed is:

1. A finished pharmaceutical dosage form containing no more than twenty-five milligrams of an active pharmaceutical ingredient and at least one excipient wherein the active pharmaceutical ingredient comprises no more than fifty percent of the mass of the finished pharmaceutical dosage form and wherein the active pharmaceutical ingredient was blended and milled with the at least one excipient to create a milled blend prior to preparation of the finished pharmaceutical dosage form comprising the milled blend.

2. The finished pharmaceutical dosage form in claim 1,
wherein the active pharmaceutical ingredient is ivermectin.

3. The finished pharmaceutical dosage form in claim 1,
wherein the active pharmaceutical ingredient is ivermectin; and
wherein the amount of ivermectin is about two milligrams.

4. The finished pharmaceutical dosage form in claim 1,
wherein the active pharmaceutical ingredient is ivermectin; and
wherein the amount of ivermectin is ten milligrams or less.

5. The finished pharmaceutical dosage form in claim 1,
wherein the active pharmaceutical ingredient is at least one hormonal steroid.

6. A finished pharmaceutical dosage form containing no more than twenty-five milligrams of an active pharmaceutical ingredient and at least one excipient wherein the active pharmaceutical ingredient comprises no more than fifty percent of the mass of the finished pharmaceutical dosage form and wherein the active pharmaceutical ingredient was blended and milled with the at least one excipient to create a milled blend, which is then blended with a lubricant to create a lubricated milled blend prior to preparation of the finished pharmaceutical dosage form comprising the lubricated milled blend.

7. The finished pharmaceutical dosage form in claim 6,
wherein the active pharmaceutical ingredient is ivermectin.

8. The finished pharmaceutical dosage form in claim 6,
wherein the active pharmaceutical ingredient is ivermectin; and
wherein the amount of ivermectin is about two milligrams.

9. The finished pharmaceutical dosage form in claim 6,
wherein the active pharmaceutical ingredient is ivermectin; and
wherein the amount of ivermectin is ten milligrams or less.

10. The finished pharmaceutical dosage form in claim 6,
wherein the active pharmaceutical ingredient is at least one hormonal steroid.
